(19) United States
(12) Patent Application Publication
(10) Pub. No.: US 2018/0345022 A1
(43) Pub. Date: Dec. 6, 2018

(54) Enhanced Selectivity and Modulation in Coordinated Reset in Deep Brain Stimulation

(71) Applicant: Boston Scientific Neuromodulation Corporation, Valencia, CA (US)

(72) Inventors: G. Karl Steinke, Valencia, CA (US); Hemant Bokil, Santa Monica, CA (US)

(21) Appl. No.: 15/990,325

(22) Filed: May 25, 2018

Related U.S. Application Data
(60) Provisional application No. 62/514,302, filed on Jun. 2, 2017.

(51) Int. Cl. A61N 1/36; A61N 1/05

(52) U.S. Cl. CPC A61N 1/36167; A61N 1/36171; A61N 1/36175; A61N 1/0534

(57) ABSTRACT

Various manners are disclosed in which neurostimulation can be programmed to provide stimulation pulses designed to alter the level of synchronization in a target neural tissue, as is useful in Deep Brain Stimulation (DBS) therapy for example. Stimulation pulses are issued in pulse packets, with one or more variations added within or between pulse packets, such as variations in pulse width, amplitude, frequency, or shape. Such variations afford greater ability to differentially recruit sub-populations of neural tissue in both space and time. Such pulse packets may be issued from one or more electrodes, which pulse packets may or may not overlap in time.

### CROSS-REFERENCE TO RELATED APPLICATIONS

This is a non-provisional of U.S. Provisional Patent Application Ser. No. 62/514,302, filed Jun. 2, 2017, which is incorporated by reference in its entirety, and to which priority is claimed.

### FIELD OF THE INVENTION

The present invention relates to an improved stimulator system and its method of use, in which the stimulator is programmed to provide pulses in a manner to alter the firing of neural tissue as useful in Deep Brain Stimulation (DBS) for example.

### INTRODUCTION

Implantable stimulation devices are devices that generate and deliver stimuli to nerves and nervous tissues for the therapy of various biological disorders, such as pacemakers to treat cardiac arrhythmia, defibrillators to treat cardiac fibrillation, cochlear stimulators to treat deafness, retinal stimulators to treat blindness, muscle stimulators to produce coordinated limb movement, spinal cord stimulators to treat chronic pain, cortical and deep brain stimulators to treat motor and psychological disorders, and other neural stimulators to treat urinary incontinence, sleep apnea, shoulder subluxation, etc. The description that follows will generally focus on the use of the invention within a Deep Brain Stimulation (DBS) system, such as is disclosed in U.S. Patent Application Publication 2016/0184591. However, the present invention may find applicability in any implantable stimulator system.

As shown in FIG. 1, a DBS system typically includes an Implantable Pulse Generator (IPG) 10, which includes a biocompatible device case 12 formed of titanium for example. The case 12 typically holds the circuitry and battery 14 necessary for the IPG to function, although IPGs can also be powered via external energy and without a battery. The IPG 10 is coupled to electrodes 16 via one or more electrode leads (two such leads 18 and 20 are shown), such that the electrodes 16 form an electrode array 22. The electrodes 16 are carried on a flexible body 24, which may also house individual signal wires 26 coupled to each electrode. In the illustrated embodiment, there are eight electrodes on lead 18, labeled E1-E8, and eight electrodes on lead 20, labeled E9-E16, although the number of leads and electrodes is application specific and therefore can vary. The proximal ends of leads 18 and 20 couple to the IPG 10 using lead connectors 28, which are fixed in a header material 30 comprising an epoxy for example.

In a DBS application, as is useful in the treatment of Parkinson's disease for example, the IPG 10 is typically implanted under the patient's clavicle (collarbone), and the leads 18 and 20 are tunneled through the neck and between the skull and the scalp where the electrodes 16 are implanted through holes drilled in the skull in the left and right and side of the patient's brain, as shown in FIG. 2. In one example, the electrodes 16 may be implanted in the subthalamic nucleus (STN). The electrodes may be implanted in both of these regions in the left and right side of the brain, meaning that four leads might be necessary, as discussed in the above-referenced '591 Publication. Stimulation therapy provided by the IPG 10 has shown promise in reducing a patient's Parkinson's symptoms, in particular tremor that can occur in the patient's extremities.

FIG. 3 shows an environment in which an implant patient can be “fitted,” that is, where stimulation parameters for a patient can experimented with to hopefully find parameters that alleviate a patient's symptoms (e.g., tremor) while not introducing unwanted side effects. Stimulation is typically provided by pulses, and stimulation parameters typically include the amplitude of the pulses (whether current or voltage), the frequency and duration of the pulses, as well as the electrodes 16 selected to provide such stimulation, and whether such selected electrodes are to act as anodes (that source current to the tissue) or cathodes (that sink current from the tissue).

In FIG. 3, one or more of leads 18, 20 have been implanted in the patient's tissue 35 at a target location 36 such as the STN as described above. The proximal ends of lead(s) 18, 20 can either be connected to an IPG 10 also implanted in the tissue 35, which IPG 10 includes stimulation circuitry 31 programmed to provide stimulation to the electrodes 16 consistent with the prescribed stimulation parameters. The proximal ends of lead(s) 18, 20 can also be at least temporarily connected to an External Trial Stimulation 72, which is typically used to provide stimulation during a trial phase after the lead(s) 18, 20 are implanted but before the IPG 10 is permanently implanted. The proximal ends of lead(s) 18, 20 exit an incision 71 in the patient's tissue 35, and are connected to the ETS 72. The ETS 72 mimics operation of the IPG 10 to provide stimulation pulses to the tissue, and so also includes programmable stimulation circuitry 31. The ETS 31 allows a clinician to experiment with the stimulation parameters, and allows the patient to try stimulation for a trial period before the IPG 10 is permanently implanted.

Regardless whether trial stimulation is occurring via the ETS 72 or permanent stimulation is occurring via the IPG 10, a clinician programmer (CP) system 50 is shown that can be used by a clinician to adjust the stimulation parameters. The CP system 50 includes a computing device 51, such as a desktop, laptop, or notebook computer, a tablet, a mobile smart phone, a Personal Data Assistant (PDA)-type mobile computing device, etc. (hereinafter “CP computer”). In FIG. 3, CP computer 51 comprises a laptop computer that includes typical computer user interface means such as a screen 52, a mouse, a keyboard, speakers, a stylus, a printer, etc., not all of which are shown for convenience. Also shown in FIG. 3 are accessory devices for the CP system 50 that are usually specific to its operation as a stimulation controller, such as a communication wand 54, and a joystick 58, which can be connected to suitable ports on the CP computer 51, such as USB ports 59 for example. Joystick 58 is generally used as an input device to select various stimulation parameters (and thus may be redundant of other input devices to the CP), but is also particularly useful in steering currents between electrodes to arrive at an optimal stimulation program.

In operation, the clinician will use the user interface of the CP computer 51 to adjust the various stimulation parameters the ETS 72 or IPG 10 will provide, and such adjusted parameters can be wirelessly transmitted to the patient. Such wireless transmission can occur in different ways. The antenna used in the CP system 50 to communicate with the ETS 72 or IPG 10 can depend on the data telemetry antenna included in those devices. If the patient's ETS 72 or IPG 10 includes a coil antenna 70a or 40a, the wand 54 can likewise include a coil antenna 56a to establish communication over a near-field magnetic induction link at small distances. In this instance, the wand 54 may be affixed in close proximity to the patient, such as by placing the wand 54 in a holster, belt, or necklace wearable by the patient and proximate to the patient's ETS 72 or IPG 10.

If the ETS 72 or IPG 10 includes a far-field RF antenna 70b or 40b with longer communication distance, the wand 54, the CP computer 51, or both, can likewise include a short-range RF antenna 56b to establish communication with the ETS 72 or IPG 10. (In this example, a CP wand 54 may not be necessary if the CP computer 51 has the necessary short-range RF antenna 56b). If the CP system 50 includes a short-range RF antenna 56b, such antenna can also be used to establish communication between the CP system 50 and other devices, and ultimately to larger communication networks such as the Internet. The CP system 50 can typically also communicate with such other networks via a wired link 62 provided at a Ethernet or network port 60 on the CP computer 51, or with other devices or networks using other wired connections (e.g., at USB ports 59). Far-field RF antennas 56b, 70b, and/or 40b may operation with well-known communication standards such as Bluetooth, WiFi, ZigBee, MICS, etc.

To program stimulation parameters, the clinician interfaces with a clinician programmer graphical user interface (CP GUI) 64 provided on the display 52 of the CP computer 51. As one skilled in the art understands, the CP GUI 64 can be rendered by execution of CP software 66 on the CP computer 51, which software may be stored in the CP computer's non-volatile memory 68. One skilled in the art will additionally recognize that execution of the CP software 66 in the CP computer 51 can be facilitated by control circuitry 70 such as a microprocessor, microcomputer, an FPGA, other digital logic structures, etc., which is capable of executing programs in a computing device. Such control circuitry 70 when executing the CP software 66 will in addition to rendering the CP GUI 64 enable communications with the ETS 72 or IPG 10 as explained earlier, so that the clinician can use the CP GUI 64 to program the stimulation parameters to the stimulation circuitry 31 in the patient's ETS 72 or IPG 10. Examples of the CP GUI 64 can be found in U.S. Patent Application Publication 2015/0360038 and U.S. Provisional Patent Application Ser. No. 62/471,540, filed Mar. 15, 2017.

A hand-held, portable patient external controller 50 can also be used to adjust stimulation parameters, which may include one or both of a coil antenna 52a or an RF antenna 52b capable of communicating with the ETS 72 of IPG 10. Further details concerning an external controller 50 can be found in the above-referenced '038 Publication.

It has been hypothesized that a cause of symptoms (e.g., tremors) in DBS patients relates to an undue high degree of neural synchronicity (hyper-synchronicity) in the target neural population 36. That is, the neurons within location 36 are overly coupled to one another, and thus fire in synch, leading to symptoms. Further, a neural population 36 may also have an unduly low degree of neural synchronicity (hypo-synchronicity), which may also lead to symptoms.

A technique that may alter the synchronicity of neural firing in the target neural population 36, called coordinated reset, is shown in FIG. 4A. Coordinated reset involves using stimulation pulses at two or more electrodes Ex to stimulate different sub-populations 82(x) of neurons within the target neural population 36 at different times, as shown in FIG. 4B. For example, a first packet of pulses 80 is issued from electrode E1 during a time period t1, with the goal of causing neurons within subpopulation 82(1) to fire. Another packet of pulses 80 is issued from electrode E2 during a later time period t2, with the goal of causing neural elements (e.g., neurons, fibers, nerve terminals, etc.) within sub-population 82(2) to fire, and so on for electrodes E3 and E4 and sub-populations 36(3) and 36(4) during times t3 and t4. The pulse packets 80 can then be repeated at electrodes E1-E4 during times periods t5-t8 as shown in FIG. 4A. A gap in time may exist between successive pulse packets 80. Further, the pulse packets 80 delivered to electrodes E1-E4 occur during a time period Ts, which preferably matches the frequency fs at which the sub-populations 82(x) are noticed to oscillate, such as between 12 to 25 Hz for example.

FIG. 4B shows a generalized map which explains the degree of coupling between neural sub-populations 82(x) within the target neural population 36. Coupling can be explained by denoting a weight of coupling w_{x,y} between two sub-populations 82(x) and 82(y) proximate to electrodes Ex and Ey. (Electrodes E1-E4 may be on the same or on different leads 18, 20). When target neural tissue 36 is hyper-synchronized, the weights are too high; if hypo-synchronized, the weights are too low. Thus, when target neural tissue 36 is hyper-synchronized, firing of neurons in say sub-population 82(1) causes too easily the firing of neurons in sub-population 82(3), even if firing of these sub-populations 82(1) and 82(3) occurs at different times or phases. High neural coupling between sub-populations, even if not at the same phase, is described as “entrainment.” Likewise, when target neural tissue 36 is hypo-synchronized, firing of neurons in say sub-population 82(1) may not readily cause the firing of neurons in sub-population 82(3), even if it should.

Coordinated reset as provided by the pulse packets 80 of FIG. 4A may cause the phase of the oscillatory neural activity in the sub-populations 82(x) to be reset. For example, the time period between pulse packets, T_{T}, may be 12.5 msec. Suppose that when sub-population 82(1) fires, sub-population 82(2) will naturally fire due to high entrainment (w_{1,2}) 15 msec later when no stimulation is present. Because the pulse packet 80 at electrode E2 is issued earlier than this—at 12.5 msec—the natural coupling between sub-populations 82(1) and 82(2) is disrupted and therefore reset. In other words, issuing the pulse packets 80 during time periods t1, t2, t3, etc., is likely to disrupt the otherwise naturally high coupling and phase of firing between the sub-populations 82(x) were stimulation not used, which promotes desynchronization and assists in the reduction of symptoms. By the same token, coordinate reset as described in FIG. 4A may also assist in synchronizing undesirably hypo-synchronized sub-populations 82(x).

### BRIEF DESCRIPTION OF THE DRAWINGS

FIG. 1 shows an implantable pulse generator (IPG) with an electrode array in accordance with the prior art.

FIG. 2 shows implantation of the IPG in a patient in a Deep Brain Stimulation (DBS) application in accordance with the prior art.

FIG. 3 shows implantation of one or more leads in target neural tissue, and connection of the lead(s) to an IPG or an External Trial Stimulation (ETS). External devices for programming the stimulation circuitry in the ETS or IPG, such as a clinician's programmer and a patient external controller, are also shown.

FIG. 4A shows pulse packets issued sequentially from different electrodes in accordance with a technique called coordinated reset.

FIG. 4B shows a generalized map showing coupling between and recruitment of different neural sub-populations when the standard coordinated reset pulse therapy of FIG. 4A is used.

FIG. 5 shows a generalized map showing varied or randomized recruitment of neural populations using the disclosed techniques which add variation or randomization to the pulses in the pulse packets.

FIG. 6 shows a first example of variation or randomization, in which different pulse packets have different pulse widths as issued from a single electrode.

FIG. 7 shows a first example of variation or randomization, in which different pulse sections within a pulse packet have different pulse widths as issued from a single electrode.

FIG. 8 shows third examples of variation or randomization, in which different pulse packets have different pulse amplitudes, or in which different pulse sections within a pulse packet have different pulse amplitudes, as issued from a single electrode.

FIG. 9 shows fourth examples of variation or randomization, in which different pulse packets have different pulse shapes, or in which different pulse sections within a pulse packet have different pulse shapes, as issued from a single electrode.

FIG. 10 shows a fifth example of variation or randomization, in which different pulse packets vary in two or more of the previously described manners, as issued from a single electrode.

FIG. 11 shows a sixth example of variation or randomization, in which different pulse packets vary in two or more of the previously described manners, and issue from two or more electrodes.

FIG. 12 shows a seventh example of variation or randomization, in which the timing of the pulse packets from the two or more electrodes in FIG. 11 is randomized.

FIGS. 13A and 13B show eighth examples of variation or randomization, in which the pulse packets from the two or more electrodes in FIG. 11 overlap in whole or in part.

FIG. 14 shows a ninth example of variation or randomization, in which pulses within a pulse packet or a pulse section within a pulse packet overlap in time with constant amplitude pulses issued from another electrode.

### DETAILED DESCRIPTION

Applicant discloses various manners in which the stimulation circuitry of an IPG or ETS can be programmed to provide stimulation pulses designed to alter the synchronicity of firing in target neural tissue 36—that is, to affect desynchronicity in hyper-synchronized neural tissue or to affect synchronicity in hypo-synchronized tissue, thus resulting in alleviation of a patient's neurological symptoms. Applicant's technique can vary in a number of different ways discussed subsequently, but by way of quick review, Applicant's technique issues stimulation pulses with one or more variations to attempt to recruit and stimulate a wider variety of sub-populations of neurons in both space and time.

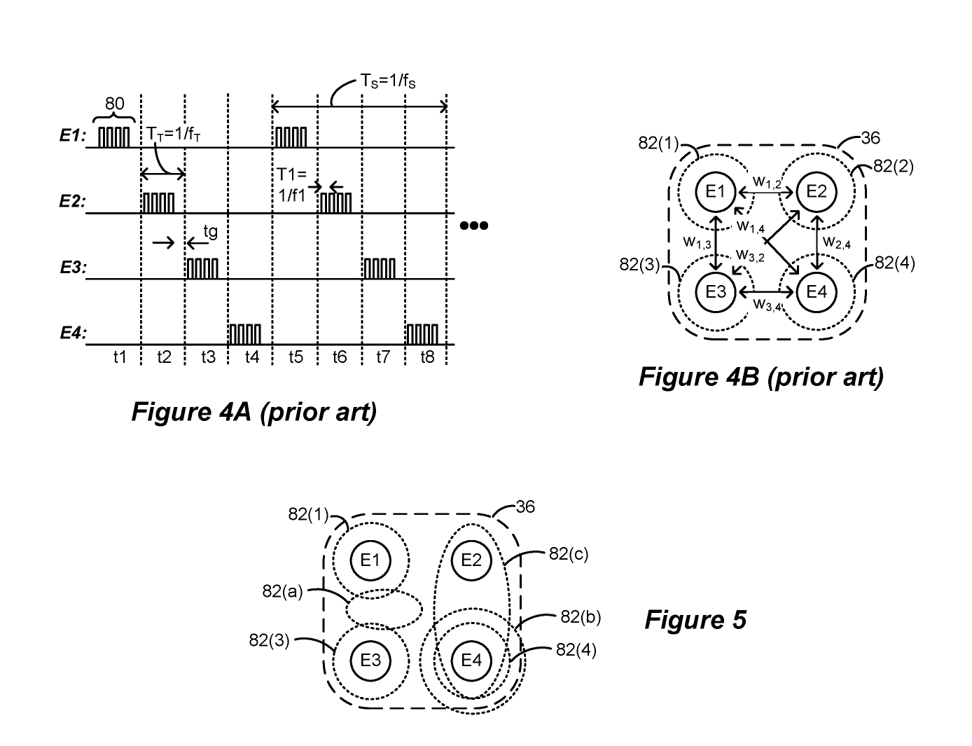

This is shown generally by comparing the standard coordinate reset map of FIG. 4B and a map illustrative of Applicant's technique in FIG. 5. Notice in FIG. 4B that because non-varying pulses are issued during pulse packets 80 at each of the electrodes with predictable timings, the sub-populations 82 that are recruited by the stimulation never change, and are generally proximate to (e.g., surrounding) the electrode receiving the pulses. By contrast, variations in the issued pulses using Applicant's technique recruits sub-populations 82 that can vary in space and time. For example, sub-population 82(a) in FIG. 5 includes neural elements between electrodes E3 and E1 which are not recruited (at least to the same degree) as during standard coordinated reset, and may be recruited at a time different from when sub-populations 82(1) and 82(3) are recruited. Sub-population 82(b) recruits a subpopulation around electrode E4, which differs in size from the sub-population 82(4) recruited during standard coordinated reset, and both of 82(b) and 82(4) can be recruited in Applicant's technique at different times. Sub-population 82(c) recruits at a single time a subpopulation around electrodes E2 and E4, which does not occur during standard coordinated reset, which again can occur at a time different from the recruitment of 82(2) and 82(4) individually.

Because variation and randomization as used during Applicant's technique serve to further spatially and/or temporally vary the sub-populations recruited in target neural area 36, decoupling in hyper-synchronized target neural tissue 36, or coupling in hypo-synchronized target neural tissue 36, is further promoted.

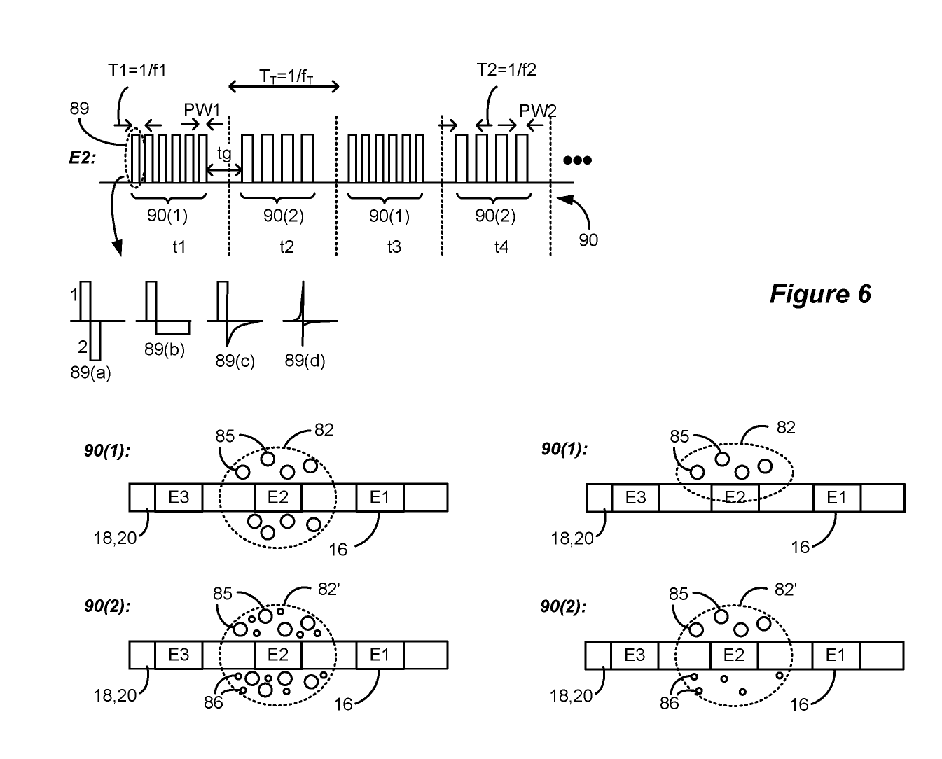

FIG. 6 shows a first variation, and it and subsequent figures (FIGS. 6-10) also illustrate different ways in which variation may be provided by modifying stimulation pulses at a single electrode. As will be shown, such modifications can recruit different neural sub-populations 82, and so can help to alter synchronization (to promote desynchronization or synchronization) of the target neural tissue even without the additional assistance of stimulation at different electrodes. That being said, all of the single-electrode variations can also be used in conjunction with stimulation at other electrodes, as discussed subsequently.

In the example of FIG. 6, the stimulation circuitry 31 has been programmed to issue successive pulse packets 90, each comprising a number of individual pulses 89. The pulses packets 90 comprise two types, 90(1) and 90(2) having different pulse widths PW1 and PW2 respectively. The pulses in pulse packets 90(1) and 90(2) may also issue with different frequencies f1 and f2 as well. As shown, the pulse packets of longer (90(1)) and shorter (90(2)) pulse widths are alternated, and are issued from a single electrode, such as E2 for example. The electrode E2 can be determined by the clinician or patient as an electrode that when stimulated produces desirable therapy, and stimulation circuitry 31 can thus be programmed to route the stimulation pulses to that electrode or any other that might provide therapeutic results. Two different pulse widths and/or frequency are shown for simplicity, but pulse packets with three or more pulse widths and or frequencies could be provide as well. To give some non-limiting examples, pulse widths PW1 and PW2 may range from 10 microseconds to 1 milliseconds, and frequencies f1 and f2 may be as high as 20 kHz.

The individual pulses 89 within the pulse packets are shown for simplicity as constant current or constant voltage uniphasic pulses having a single (positive) phase). However, in an actual example, charge recovery may be implemented, which would change the shapes of the pulses. Different examples of pulses 89 are shown in FIG. 6. The first two, 89a and 89b, comprise a biphasic pulse comprising a first actively-drive phase (1) and a second actively-driven phase (2) of the opposite polarity. This reversal of the polarity of the current during the second phase assist in recovering any stray charge that may linger on structures (capacitances) at the end of the first phase, as is known. See, e.g., U.S. Patent Application Publication 2016/0144183. The first and second phases may have the same pulse widths (89(a)), or different sized pulse widths (89(b)), and it is preferable that the same amount of charge is represented in both phases to ensure perfect recovery. Pulse 89(c) uses active recovery following the active pulse phase, as shown by the opposite polarity exponential decay, which passive recovery can involve shorting the electrode after issuance of the pulse, as explained in the '183 Publication. Pulse 89(d) shows another pulse shape representing a spike or delta function, whose amplitude is not constant over its duration, and may be issued from the discharge of a capacitor in the stimulation circuitry 31 for example.

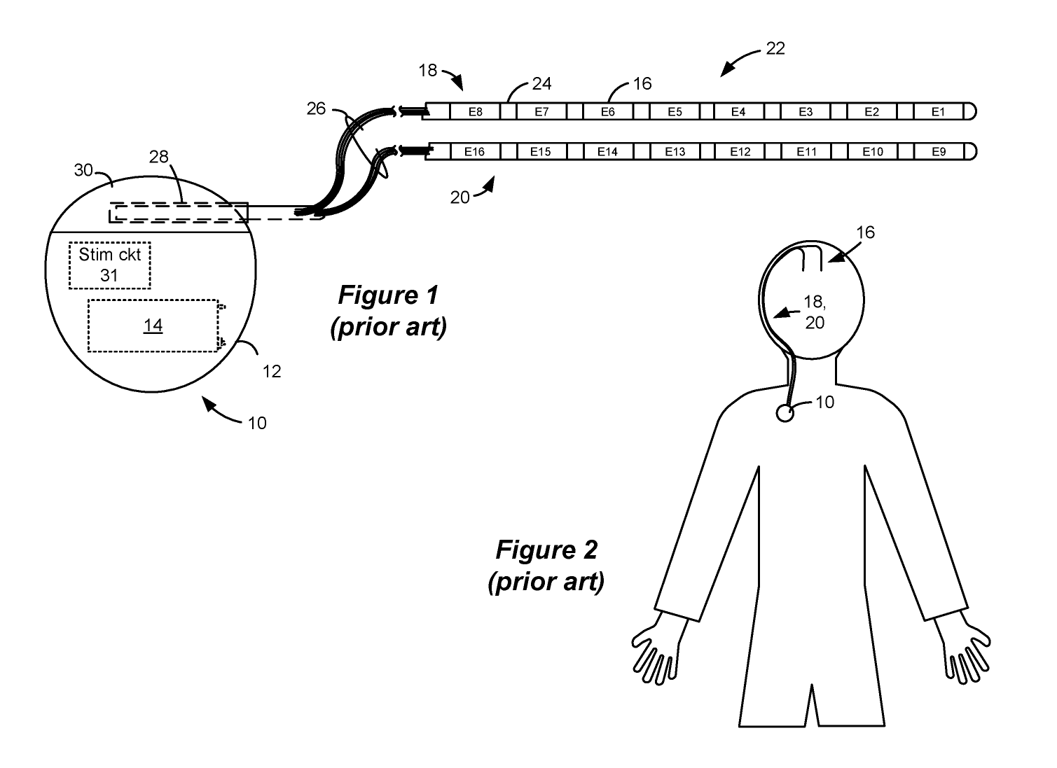

Although not shown, each of the pulses 89 may be referenced to different return electrodes for the currents that they source or sink from the tissue. For example, the conductive case 12 (FIG. 1) may comprise the return electrode for the current, and the case 12 may be actively driven by source or sink circuitry to match the current provided by E2 (but with opposite polarity). Alternatively, the case 12 may comprise a passive electrode held at a nominal potential (e.g., ground). Use of the case 12 as a return electrode is commonly referred to as monopolar stimulation. Bipolar or multipolar situation may also be used, in which one or more electrodes on the lead(s) 18, 20 are used as the return electrode for the current. For example, electrode E1 or E3 could be used as the return electrode for pulses provided at electrode E2 (bipolar stimulation), or both could be used (tripolar stimulation). As with the case 12, such lead-based return electrodes can be actively driven, or can comprise passive electrodes.

Because different pulse widths PW1 and PW2 are used in the different pulse packets 90(1) and 90(2), different neural sub-populations will be recruited within the target neural tissue 36, as shown in the drawings at the bottom of FIG. 6. As shown to the left, the issuance of smaller pulse width pulses (PW1, 90(1)) will recruit within subpopulation 82 larger-diameter neural elements 85 within subpopulation 82(2) proximate to the stimulated electrode E2. By contrast, the issuance of longer pulse widths (PW2, 90(2)) will recruit within subpopulation 82′ both larger-diameter neural elements 85 and smaller-diameter neural elements 86. See https://en.wikipedia.org/wiki/Rheobase#Strength-Duration_Curve. Larger- and smaller-diameters neural elements 85 and 86 will fire at different rates, and so modification of the pulse widths during pulse packets 90(1) and 90(2) will randomize that firing of subpopulation 82.

Modifying the pulse width may also affect the spatial distribution of the recruited sub-population. As shown in the drawings at the bottom right in FIG. 6, larger-diameter neural elements 85 may exist on only one side of the stimulated electrode, thus resulting in a first recruited sub-population 82 when short pulse widths (PW1, 90(1)) are used. However, smaller-diameter neural elements 86 may exist on the other side of the stimulated electrode, thus resulting in a second recruited sub-population 82 when large pulse widths (PW2, 90(2)) are used. In any event, modifying the pulse width of the pulses affects and can be used to modulate selected sub-populations to alter the synchronicity within the target neural population.

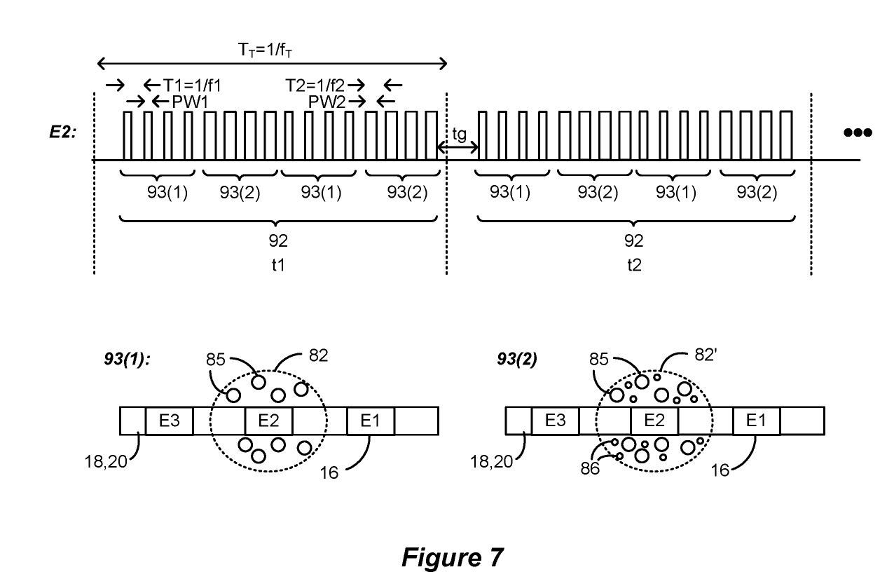

FIG. 7 also involves modification of the pulse width of the pulses, but in this example the pulse width is modified inside of each pulse packet 92. Thus, each pulse packet 92 includes pulse sections 93(1) having smaller pulse widths (PW1) and pulse sections 93(2) having longer pulse widths 93(2). This varies the recruited subpopulations 86 and 86′ proximate to the simulated electrode during the issuance of each of these pulse sections 93(1) and 93(2), similar what was described earlier with reference to FIG. 6. Although not shown, it should be understood that the number of pulses in each pulse section 93(1) and 93(2) could be varied or randomized to further promote randomization of the recruited subpopulation, and hence alter firing with the target neural population. Again, different numbers of pulse section 93(x) could be used, as well as different frequencies for the pulses in each pulse section.

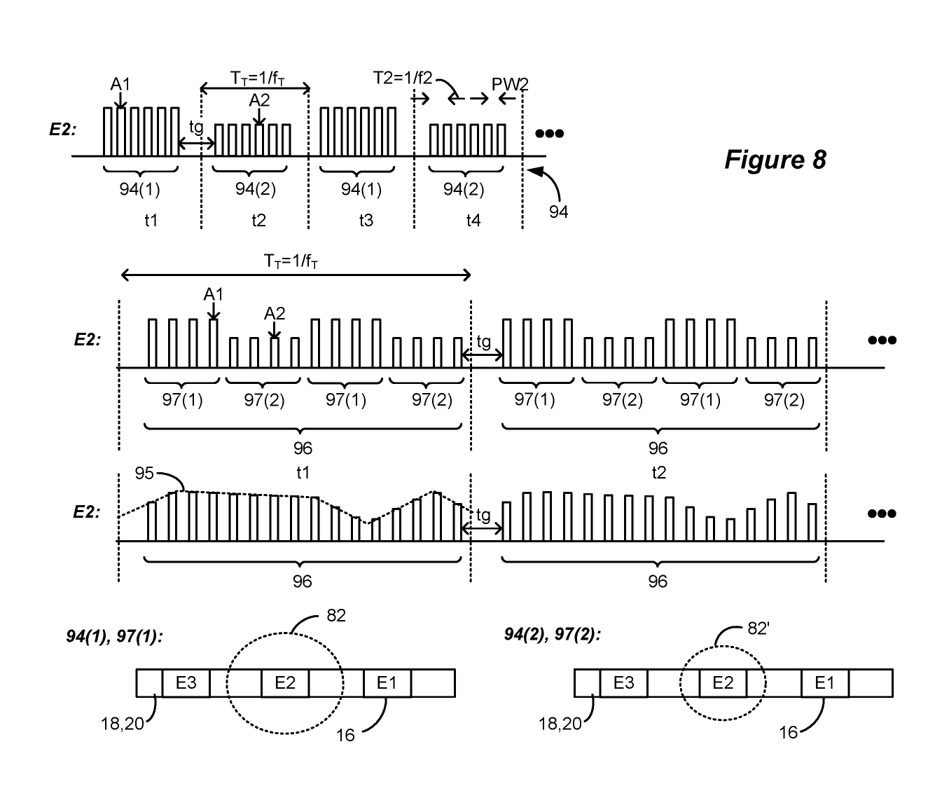

FIG. 8 involves modification to the amplitude of the pulses to affect different sub-population recruitment. In the top waveform of FIG. 8, alternating pulse packets 94(1) and 94(2) are issued by the stimulation circuitry 31 with (current or voltage) amplitudes A1 and A2 respectively. In the middle waveform, amplitude adjustment occurs within each pulse packet 96, which has pulse sections 97(1) and 97(2) with the different amplitudes A1 and A2. The bottom waveform shows that the amplitude of the pulses can be randomized and may include more than two values. For example, the amplitude of the pulses in the pulse packets 96 in the bottom waveform are seen to follow an amplitude curve 95.

Varying the amplitude of the pulses between pulse packets 94(1) and 94(2), or within pulse packet 96 (97(1) and 97(2)) affects and varies the subpopulation of neural elements that are recruited proximate to the stimulated electrode, and so too can be used as variable that alters synchronization in the target neural tissue 36. This is shown at the bottom of FIG. 8, in which large amplitude pulses packets 94(1) or pulse sections 97(1) within pulse packet 96 are shown to recruit a larger sub-population 82, while small amplitude pulses packets 94(2) or pulse sections 97(2) recruit a smaller sub-population 82′.

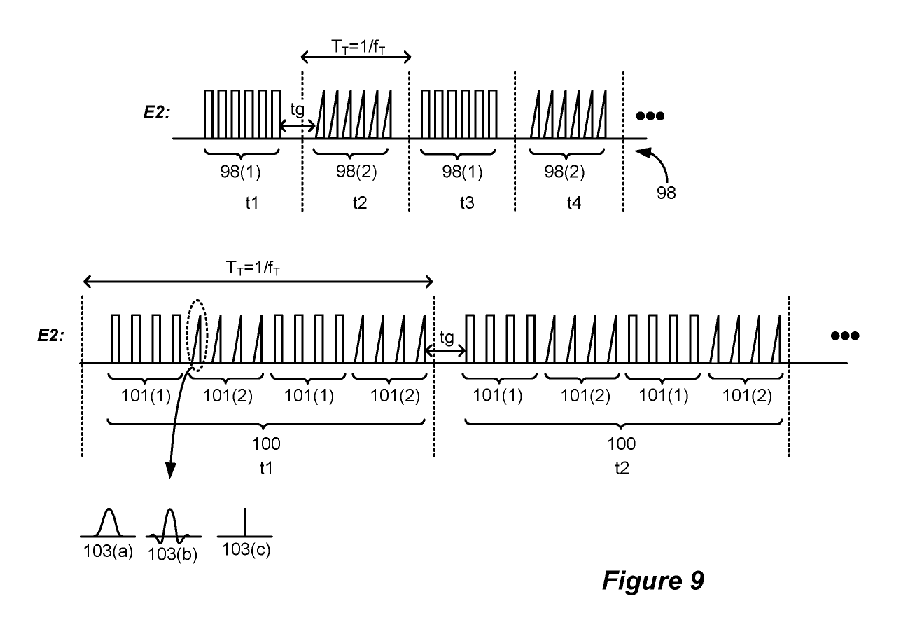

FIG. 9 involves modification to the shape of the pulses to affect different sub-population recruitment. In the top waveform of FIG. 9, alternating pulse packets 98(1) and 98(2) are issued by the stimulation circuitry 31 with different shapes. Square pulses (98(1)) and ramped pulses (98(2)) are shown, but other pulse shapes could be used as well, some of which 103(a)-(c) are also shown at the bottom of FIG. 9. In the bottom waveform, pulse shape adjustment occurs within each pulse packet 100, which has pulse sections 101(1) and 101(2) with different shaped pulses. More than two pulses shapes could be used in pulse packets 98 or 100, and the number of each pulse or their positions in the packets could be randomized. As one skilled in the art will recognize, pulses of different shapes will recruit different neural elements in the subpopulation.

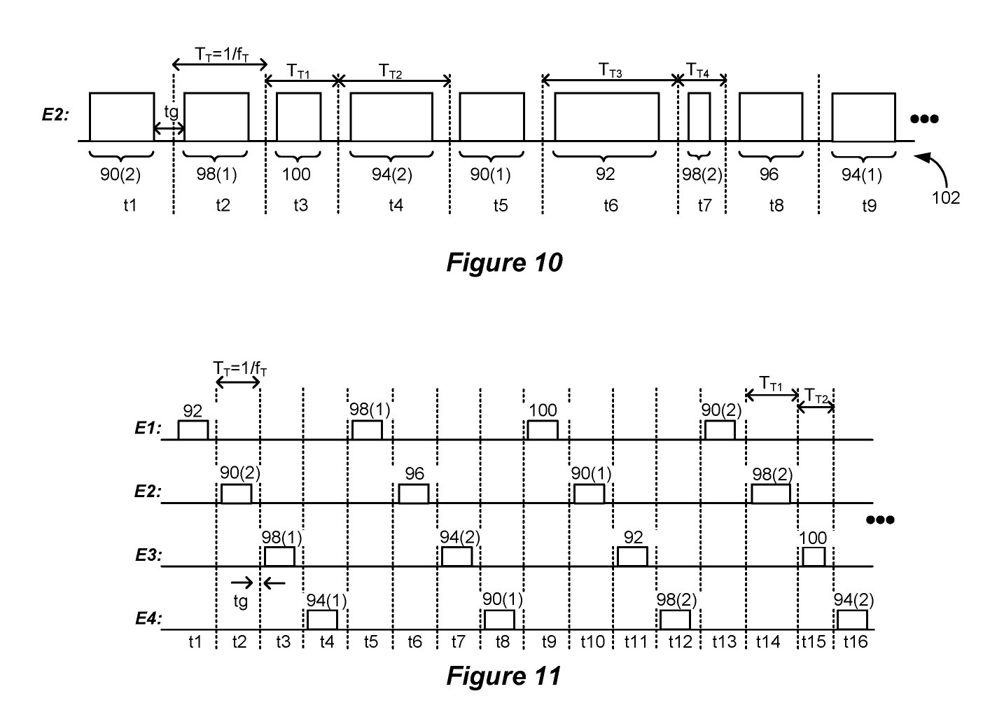

FIG. 10 shows that the pulse packets 102 issued from a selected stimulation electrode (e.g., E2) can be varied using combinations of the pulse packets described earlier in FIGS. 6 through 9. For example and as shown, a first pulse packet 90(2) with large pulse width pulses (FIG. 6) is issued; followed by a second pulse packet 98(1) with pulses of a first shape (FIG. 9); followed by a third pulse packet 100 (FIG. 9) having a mixture of different pulses shapes; followed by a fourth pulse packet 94(2) having pulses with a small amplitude (FIG. 7); etc. Adding further to the variance, and thus the ability to alter synchronization, the duration of each of the pulse packets may also be varied. Thus, while most of the pulse packets last for a duration of T_{T}, some pulse packets are issued with smaller (T_{T1}, T_{T4}) or longer (T_{T2}, T_{T3}) durations.

While FIGS. 6-10 show techniques in which pulses from a single electrode can be varied to vary recruitment of a sub-population within a target neural population 36, further benefits are had when the techniques are used at two or more electrodes, as occurs in the standard coordinated reset technique described earlier (FIGS. 4A and 4B). For example, in FIG. 11, pulse packets are issued from four electrodes E1-E4 at discrete non-overlapping time periods generally equal to T_{T}. A gap tg appears between the time periods as described earlier. However, the pulse packets do not comprise the same pulses, but instead vary in the various ways described earlier. For example, the first pulse packet 92 issued from E1 (t1) includes pulses with varying pulse widths and possibly with varying frequencies as well (FIG. 7); the second pulse packet 90(2) issued from E2 (t2) includes pulses with larger pulse widths (FIG. 6); the third pulse packet 98(1) issued from E3 (t3) includes pulses of a first shape (FIG. 9); the fourth pulse packet 94(1) issued from E3 (t4) includes pulses of a larger amplitude (FIG. 8); the fifth pulse packet 98(1) issued from E1 (t5) again includes pulse of the first shape (FIG. 9), but later pulse packets 98(2) of a different pulse shape are issued at different times (t12, t14). Varying the pulses packets in these manners again randomizes recruitment of the sub-populations and alters synchronization. Further, and as shown at the end of FIG. 11, the duration of the pulses packets may be varied to further randomize sub-population recruitment, as shown by the issuance of a longer pulse packet 98(2) (T_{T1}), and a shorter pulse packet 100 (T_{T2}).

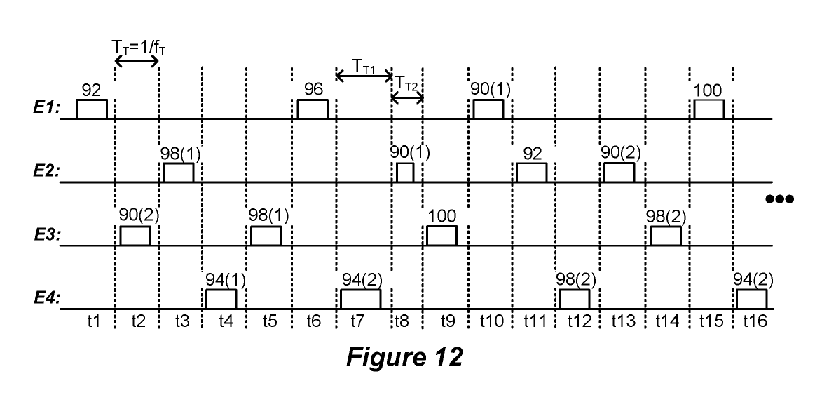

FIG. 12 shows another example similar to FIG. 11 in which various types of pulses packets 90-100 are issued from a plurality of electrodes. However, in this example, the pulse packets are not issued in a predictable order from the electrodes. As shown, the pulse packets are issued sequential from E1 (t1), E3 (t2), E2 (t3), E4 (t4), E3 (t5), E1 (t6), E4 (T7), etc. Further, as discussed above with respect to FIG. 10, the time periods T_{T} may be different for the various packets (see, e.g., T_{T1}, T_{T2}).

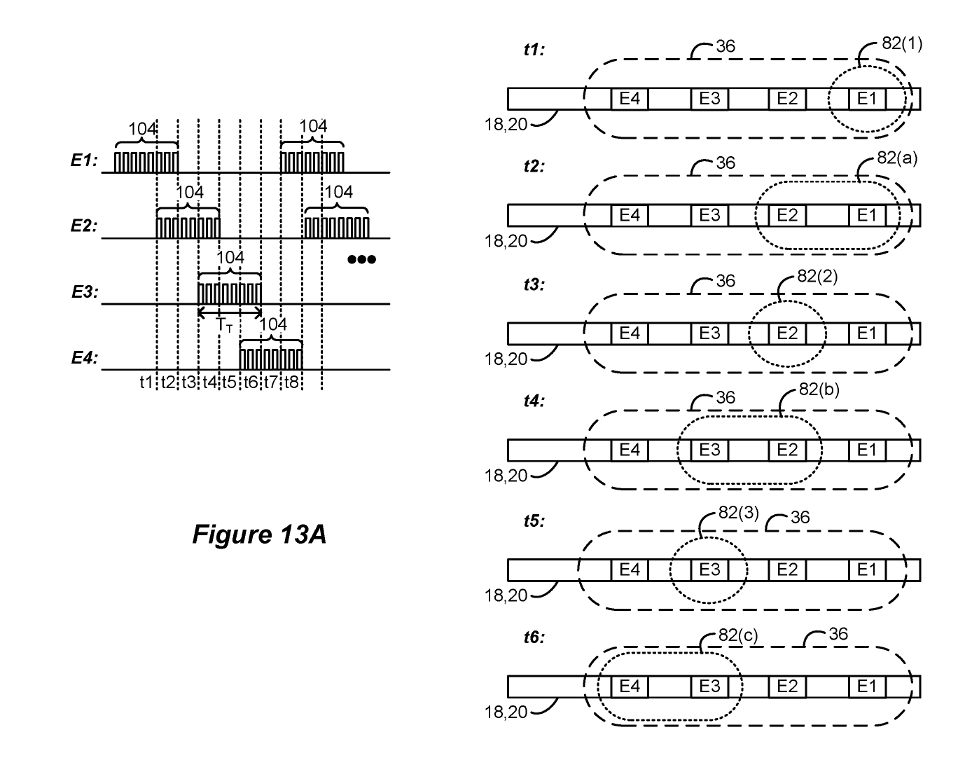

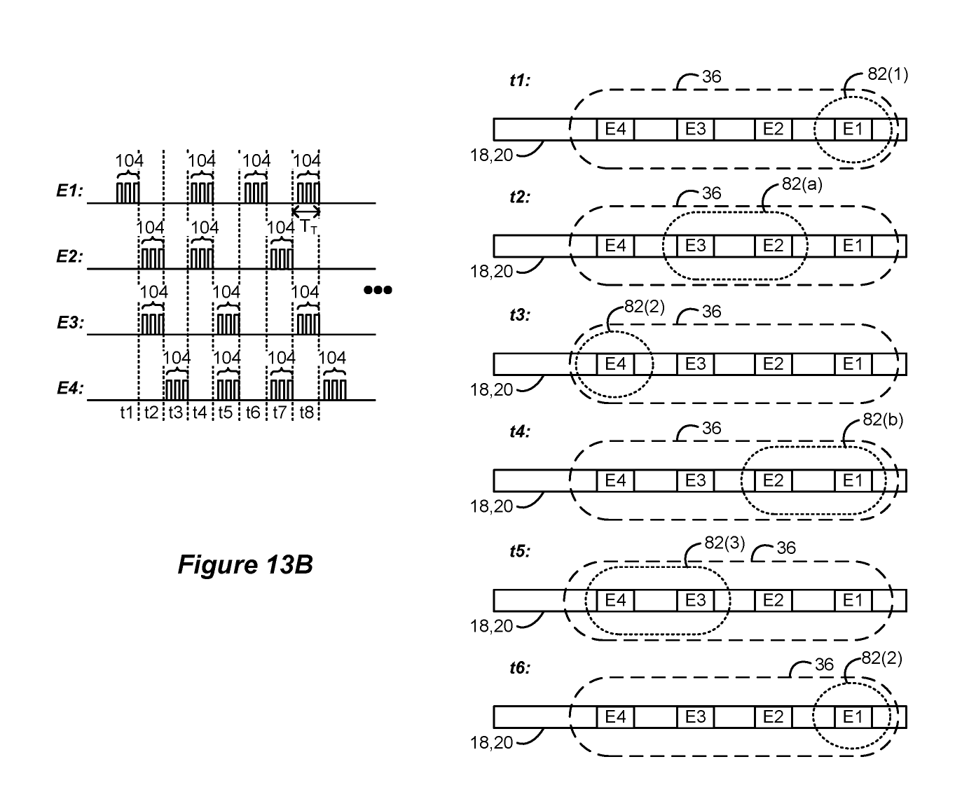

FIGS. 13A and 13B show another example in which various types of pulses packets 104 (i.e., any of pulse packets 80 or 90-100 described earlier) are issued from a plurality of electrodes. However, in these examples, the pulse packets overlap in time. Thus, in FIG. 13A, a first pulse packet 104 is issued from electrode E1 during time periods t1 and t2. A second pulse packet 104 is issued from 2 during time periods t2-t4. A third pulse packet 104 is issued from t4-t6, etc. Thus, pulses are issued only from electrode E1 during t1, recruiting sub-population 82(1) within target neural tissue 36 proximate to E1. Pulses are issued from electrodes E1 and E2 during t2, recruiting sub-population 82(a) proximate to both E1 and E2. Pulses are issued only from electrode E2 during time t3, recruiting sub-population 82(2) within target neural tissue 36 proximate to E2. Pulses are issued from electrodes E2 and E3 during t4, recruiting sub-population 82(b) proximate to both E2 and E3, etc. Thus, even though only four electrodes are used to provide pulses in this example, eight different sub-populations (82(1)-82(4) and 82(a)-(d)) are recruited at different times, which provides further variation and thus alters the synchronicity of neural firing. As illustrated, the individual pulses in overlapping pulse packets 104 also overlap in time, but this is not strictly necessary. Although not shown, the duration of each of the pulse packets 104 (T_{T}) may also be varied or randomized, and the pulse packets may also not issue in a predictable order from the electrodes.

FIG. 13B shows another example in which certain pulse packets 104 are issued such that they completely overlap with other pulse packets in time. Further, the issuance of the pulse packets 104 are not issued from the electrodes in a predictable order. This provides both spatial and temporal randomness to the recruited subpopulations 82, as shown in the figures to the right. Again, the individual pulses in overlapping pulse packets 104 may also overlap in time or not, and the duration of each of the pulse packets 104 (T_{T}) may also be varied or randomized.

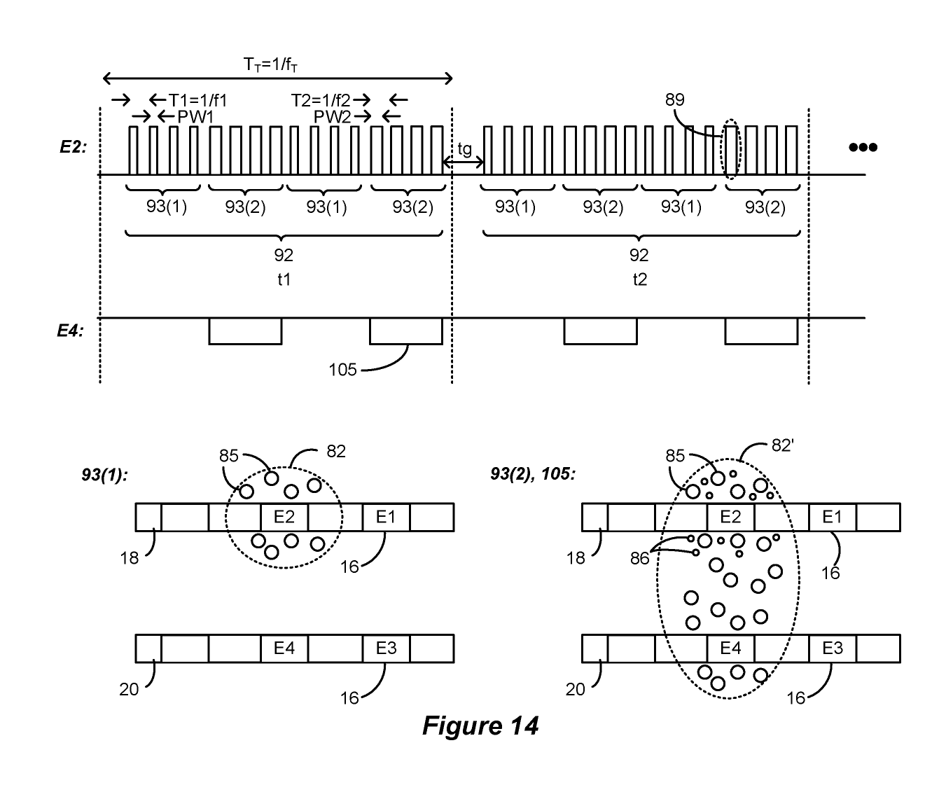

FIG. 14 shows another example in which pulse packets with higher frequency pulses 89 are issued from one electrode (E2), while constant amplitude pulses 105 are concurrently issued from another electrode (E4). In this example, the chosen electrodes are on different leads 18, 20, although this is not strictly necessary. The pulse packet 92 used for E2 as illustrated comprises the same pulse packet 92 described earlier (FIG. 7), and comprises pulse sections 93(1) having smaller pulse widths (PW1) and pulse sections 93(2) having longer pulse widths (PW2). (Separate pulse packets 90(1) and 90(2) with pulses of different pulses (FIG. 6) could also be used). The constant amplitude pulses 105 are coincident with pulse portions 93(2) having the longer pulse width pulses, and are of opposite polarity to the pulses 89. During pulse sections 93(1), there is no opposite polarity constant amplitude pulse 105; because relatively small pulse widths PW1 are used during pulse section 93(1) on electrode E2, relatively large neural elements 85 are recruited in subpopulation 82 proximate to E2, as discussed earlier (FIG. 6). During pulse sections 93(2), relatively large pulse widths PW2 are used which recruit relatively large and small neural elements 85 and 86 proximate to electrode E2, again as described earlier. However, in addition, the opposite polarity constant amplitude pulses 105 issued concurrently with pulse sections 93(2) causes hyperpolarization in larger neural elements 85 proximate to electrode E4, which are also recruited. Thus, in sum, during pulse sections 93(2) and 105, a subpopulation 82′ is recruited that is proximate to both, but in which the density of larger and smaller neural elements 85 and 86 vary in density across the recruited sub-population. This also adds variance, leading to altered synchronicity.

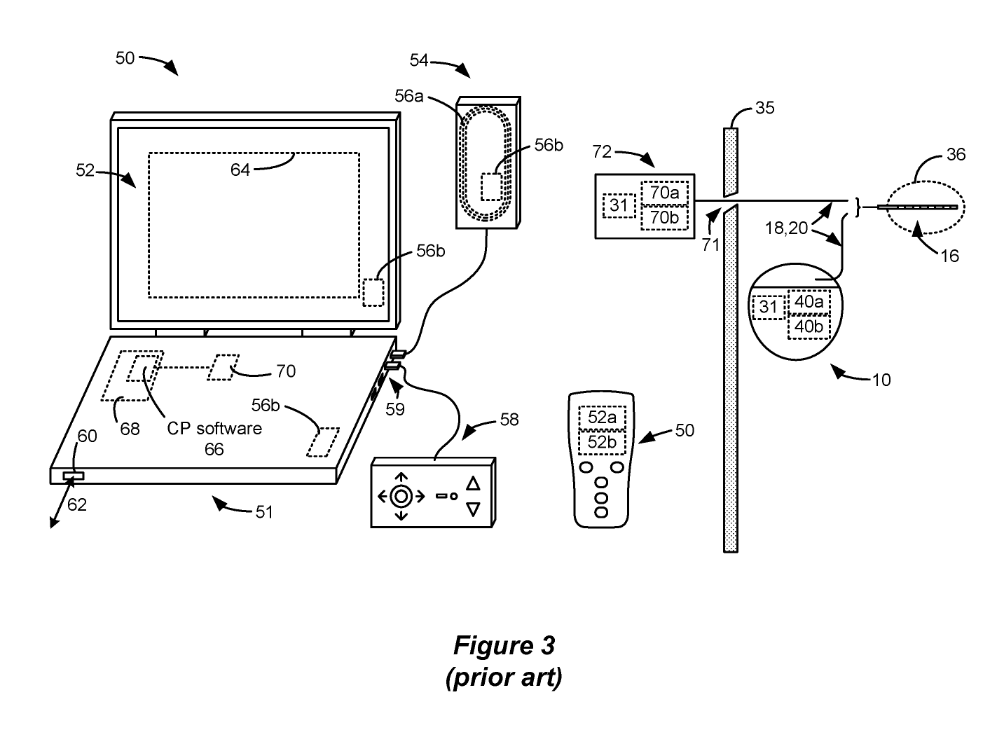

An example of stimulation circuitry 31 (FIG. 3) in either or both the ETS 72 or IPG 10 that can be programmed to produce the various types of stimulation pulses described herein can for example comprise the circuitry described in U.S. Patent Application Publications 2018/0071513 and 2018/0071520, which are both incorporated herein by reference.

Additionally, the various types of stimulation pulses described herein can be first formulated and stored as instructions in a computer-readable media associated with the clinician programmer system 50 described earlier with respect to FIG. 3, such as in a magnetic or solid state memory. Such pulses once formulated and stored can then be wirelessly transmitted to the IPG 10 as described earlier, where they are then programmed into the IPG 10's stimulation circuitry 31 for execution to form the pulses at the electrodes. The computer-readable media with such stored instructions may also comprise a device readable by the clinician programmer system 50, such as in a memory stick or a removable disk, and may reside elsewhere. For example, the computer-readable media may be associated with a server or any other computer device, thus allowing instructions for forming the disclosed stimulation pulses to be downloaded to the clinician programmer system 50 or to the IPG 10 via the Internet for example.

While benefits of the disclosed techniques focus on use in Deep Brain Stimulation (DBS) therapy, the techniques are not so limited. For example, the techniques can be used in Spinal Cord Stimulation (SCS) therapy, in which one or more leads are implanted in the epidural space within the spinal column. Other neurostimulation therapies involving neural recruitment will also benefit from the disclosed techniques.

Although particular embodiments of the present invention have been shown and described, it should be understood that the above discussion is not intended to limit the present invention to these embodiments. It will be obvious to those skilled in the art that various changes and modifications may be made without departing from the spirit and scope of the present invention. Thus, the present invention is intended to cover alternatives, modifications, and equivalents that may fall within the spirit and scope of the present invention as defined by the claims.

What is claimed is:

1. A stimulator device comprising stimulation circuitry configured to issue therapeutic pulses to at least two electrodes configured for contacting a tissue of a patient,
wherein the stimulation circuitry is configured to periodically issue, in sequence:
a first pulse packet from one of the plurality of electrodes, wherein the first pulse packets comprise a first plurality of pulses having first pulse parameters; and
a second pulse packet from another of the plurality electrodes, wherein the second pulse packets comprise a second plurality of pulses having second pulse parameters different from the first pulse parameters.

2. The stimulator device of claim 1, wherein the first and second pulse parameters comprise at least one of amplitude, pulse width, frequency, or pulse shape.

3. The stimulator device of claim 1, wherein the first and second pulse packets have different durations.

4. The stimulator device of claim 1, wherein the first and second pulse packets have randomized durations.

5. The stimulator device of claim 1, wherein the first and second pulse packets are periodically issued at random ones of the plurality of electrodes.

6. The stimulator device of claim 1, wherein the first pulse packets are issued from a first of the plurality of electrodes, and wherein the second packets are issued from a second of the plurality of electrodes.

7. The stimulator device of claim 1, wherein the first and second plurality of pulses are biphasic pulses.

8. The stimulator device of claim 1, wherein the first plurality of pulses vary in amplitude during the first pulse packet.

9. The stimulator device of claim 1, wherein the first and second pulse packets do not overlap in time.

10. The stimulator device of claim 1, wherein at least some of the first and second pulse packets overlap in time.

11. The stimulator device of claim 10, wherein all of the first and second pulse packets overlap in time.

12. The stimulator device of claim 1, wherein the stimulation circuitry is further configured to periodically issue, in sequence, a third pulse packet from yet another of the plurality electrodes, wherein the third pulse packets comprise a third plurality of pulses having third pulse parameters different from the first or second pulse parameters.

13. A stimulator device comprising stimulation circuitry configured to issue therapeutic pulses to at least two electrodes configured for contacting a tissue of a patient,
wherein the stimulation circuitry is configured to periodically issue, in sequence:
a first pulse packet from one of the plurality of electrodes, wherein the first pulse packets comprise a plurality of sequential first pulse sections, wherein the first pulse sections comprise a plurality of pulses having pulse parameters, and wherein the pulse parameters vary between different ones of the sequential first pulse sections; and
a second pulse packet from another of the plurality of electrodes, wherein the second pulse packets comprise a plurality of sequential second pulse sections, wherein the second pulse sections comprise a plurality of pulses having pulse parameters, and wherein the pulse parameters vary between different ones of the sequential second pulse sections.

14. The stimulator device of claim 13, wherein the pulse parameters comprise at least one of amplitude, pulse width, frequency, or pulse shape.

15. The stimulator device of claim 13, wherein the first and second pulse packets have different or randomized durations.

16. The stimulator device of claim 13, wherein the first and second pulse packets are periodically issued at random ones of the plurality of electrodes.

17. The stimulator device of claim 13, wherein the first pulse packets are issued from a first of the plurality of electrodes, and wherein the second packets are issued from a second of the plurality of electrodes.

18. The stimulator device of claim 13, wherein the plurality of pulses are biphasic pulses.

19. The stimulator device of claim 13, wherein the first and second pulse packets do not overlap in time.

20. The stimulator device of claim 13, wherein at least some of the first and second pulse packets overlap in time.
